(19) United States
(12) Patent Application Publication
(10) Pub. No.: US 2019/0151551 A1
(43) Pub. Date: May 23, 2019

(54) DELIVERY DEVICE WITH A CONNECTOR HAVING DISPLACEABLE PORTIONS

(71) Applicant: AbbVie Inc., North Chicago, IL (US)

(72) Inventor: Ian Cleathero, Melton Mowbray (GB)

(21) Appl. No.: 16/184,997

(22) Filed: Nov. 8, 2018

Related U.S. Application Data
(60) Provisional application No. 62/584,507, filed on Nov. 10, 2017.

(51) Int. Cl. A61M 5/24; A61M 5/20; A61M 5/315

(52) U.S. Cl. CPC A61M 5/2422; A61M 5/20; A61M 2005/2433; A61M 5/31528; A61M 5/2459

(57) ABSTRACT

A delivery device for delivering a medicament to a patient includes a cartridge disposable within a housing having a first end sealed by a septum and a second open end longitudinally opposed to the first end and defining a cartridge volume for holding the medicament. A stopper is at least partially disposed within the cartridge volume and displaceable within the cartridge volume to eject the medicament from the cartridge through the septum. A driving assembly connects to the stopper having a rotatable portion cooperatively engaged to an extendable portion such that rotation of the rotatable portion causes axial translation of the extendable portion and a connector assembly has a first connector portion connected to the extendable portion and a second connector portion connected to the stopper, the first connector portion and second connector portion are rotationally locked and axially displaceable relative to one another.

CROSS-REFERENCE TO RELATED PATENT APPLICATIONS

This application claims priority to U.S. Provisional Patent Application No. 62/584,507, filed on Nov. 10, 2017, the content of which is hereby incorporated by reference in its entirety.

### TECHNICAL FIELD

The present disclosure relates to a delivery device for administering a drug product, such as a medicament, to a patient.

### SUMMARY

The present disclosure provides improved delivery devices and components thereof. In some exemplary embodiments, the delivery device is an external drug pump. In other exemplary embodiments, the delivery device is an automatic injector, such as a wearable autoinjector.

### BRIEF DESCRIPTION OF THE FIGURES

The foregoing and other objects, features and advantages of the exemplary embodiments will be more fully understood from the following description when read together with the accompanying drawings, in which:

FIG. 1 is a side view of a prior art cartridge and driving assembly coupled together prior to expelling fluid from the cartridge.

FIG. 2 is a side view of the prior art cartridge and driving assembly shown in FIG. 1 after expelling fluid from the cartridge.

FIG. 3 is a side view of the prior art driving assembly shown in FIGS. 1-2.

FIG. 4 is a partially exploded view of the prior art driving assembly shown in FIGS. 1-3.

FIG. 5 is a side view of a prior art cartridge and driving assembly coupled together such that no gap is formed between a gear ring of the driving assembly and the cartridge.

FIG. 6 is a side view of a prior art cartridge and driving assembly coupled together such that a gap is formed between a gear ring of the driving assembly and the cartridge.

FIG. 7 is a perspective view of an exemplary embodiment of a first connector portion of a connector assembly.

FIG. 8 is a perspective view of an exemplary embodiment of a second connector portion of the connector assembly.

FIG. 9 is another perspective view of the second connector portion shown in FIG. 8.

FIG. 10 is a perspective view of an exemplary embodiment of an assembled connector assembly including the first connector portion shown in FIG. 7 and the second connector portion shown in FIGS. 8-9 in an extended position.

FIG. 11 is a cross-sectional view of the connector assembly shown in FIG. 10 taken along line 11-11

FIG. 12 is a perspective view of the connector assembly shown in FIGS. 10-11 after the connectors have axially displaced relative to one another to a compressed position.

FIG. 13 is a cross-sectional view of the connector assembly shown in FIG. 12 taken along line 13-13.

FIG. 14 is a side view of a cartridge with a stopper disposed therein connected to a driving assembly by the connector assembly shown in FIGS. 10-13 such that a gap is initially formed between a gear ring of the driving assembly and the cartridge and the connector assembly is in the extended position.

FIG. 15 is a side view of the connected stopper and driving assembly shown in FIG. 14 after the gap between the gear ring and cartridge has closed and changed the connector assembly to the compressed position.

### DETAILED DESCRIPTION

The present disclosure provides delivery devices, components thereof, and methods for injecting a medicament into a patient.

The apparatus and methods presented herein can be used for injecting a variety of medicaments into a patient. In one embodiment, the delivery device can be configured in the form of a drug pump, i.e., a pump for infusing medicaments into a patient through a cannula. In some embodiments, the drug pump can be an infusion pump configured to deliver the medicament in a cartridge over a prolonged time period greater than one minute. In other exemplary embodiments, the delivery device can be configured in the form of an automatic injector, i.e., an injection device that automatically injects medicament into a patient upon activation. In some embodiments, the automatic injector can include one or more biasing members, such as springs, that are activated to deploy a needle and depress a plunger of the injector to carry out the automatic injection. In some embodiments, the automatic injector can be a wearable autoinjector, such as an on body delivery system that may adhere to the skin of the patient.

As used herein, the term “medicament” refers to a composition intended for use in medical diagnosis, cure, treatment, or prevention of disease. A medicament may be a therapeutic agent or a combination of therapeutic agents. A medicament may include a therapeutic protein, for example, a peptide or antibody, or antigen-binding portion thereof. A medicament may include an anesthetic, steroid, and/or any other therapeutic agent(s). In one embodiment, a medicament represents a mixture of two or even more pharmacologically active agents. In some embodiments, the medicament is a liquid therapeutic agent which includes one or more biological agents, such as a protein or antibody. In one exemplary embodiment, the medicament may be insulin for infusion to manage diabetes.

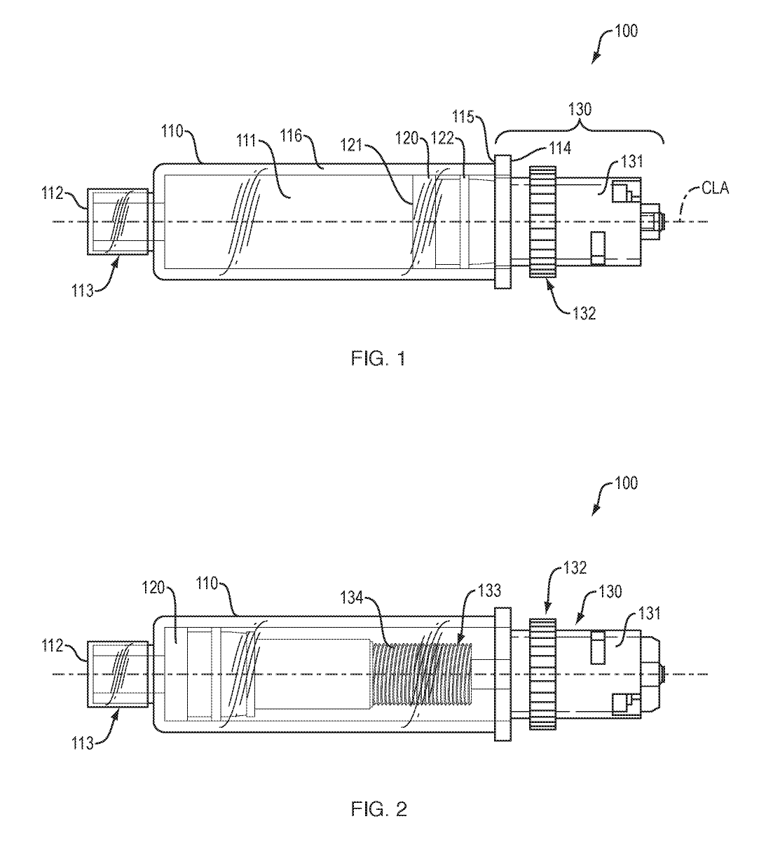

Referring now to the drawings, and more particularly to FIGS. 1-2, a delivery assembly 100 known from the prior art is shown that includes a cartridge 110 defining a cartridge volume 111, a stopper 120 disposed within the cartridge volume 111, and a driving assembly 130 connected to the stopper 120. The cartridge 110 has a first end 112 sealed by a septum 113 and a second open end 114 longitudinally opposed to the first end 112. The second open end 114 can have a rim 115. As can be seen, the cartridge 110 defines a cartridge longitudinal axis CLA and has a substantially cylindrical shape defined about the cartridge longitudinal axis CLA, but it should be appreciated that the shape of the cartridge 110 can be adjusted as desired. The cartridge 110 is dimensioned so the cartridge volume 111, which is fully or partially filled by a medicament to be delivered to a patient, can hold a desired amount of medicament. In some exemplary embodiments, the cartridge 110 is dimensioned so the cartridge volume 111 is at least 1 mL, such as between 2 and 5 mL, but the cartridge 110 can be dimensioned to have larger or smaller volumes if desired. It should thus be appreciated that the size of the cartridge 110 can be adjusted to accommodate varying volumes of medicament, corresponding to varying doses of medicament to be delivered to a patient.

The stopper 120 is disposed within the cartridge volume 111 and is axially displaceable along the cartridge longitudinal axis CLA to eject the medicament within the cartridge volume 111 from the cartridge 110. The stopper 120 may be formed of an elastomer and have a plunger portion 121 defining a first end of the stopper 120 closer to the first end 112 of the cartridge 110. The plunger portion 121, as can be seen, engages an interior surface 116 defining the boundary of the cartridge volume 111 so the plunger portion 121 can eject medicament from the cartridge 110 through the septum 113 as the stopper 120 axially displaces toward the first end 112 of the cartridge 110. The stopper 120 can also include another annular seal 122 to further guarantee sterility of the contents of the cartridge 110 and resist relative rotation between the stopper 120 and the cartridge 110.

A driving assembly 130 is connected to the stopper 120 to axially displace the stopper 120 within the cartridge volume 111 and eject medicament from the cartridge 110 through the septum 113. The driving assembly 130 can include a rotatable portion 131 having a gear ring 132 and an extendable portion 133 (best seen in FIG. 2) that is cooperatively engaged with the rotatable portion 131 such that rotation of the rotatable portion 131 causes the extendable portion 133 to axially displace. As can be seen in FIG. 2, the extendable portion 133 can have a plurality of threads 134 that are threaded to corresponding threads within the rotatable portion 131 so rotation of the rotatable portion 131 can extend or retract the extendable portion 133. It should be appreciated that while the extendable portion 133 is shown and described as being threaded to the rotatable portion 131 to convert rotation of the rotatable portion 131 into axial (linear) displacement of the extendable portion 133, the driving assembly 130 can be configured in other ways that convert rotation of the rotatable portion 131 into linear displacement of the extendable portion 133, such as a rack and pinion arrangement. The rotatable portion 131 may be rotated, for example, by a rotary motor that engages the gear ring 132. In some exemplary embodiments, the delivery assembly 100 and rotary motor can be housed together in a housing to form a fluid delivery system, which may also be referred to as a fluid delivery device.

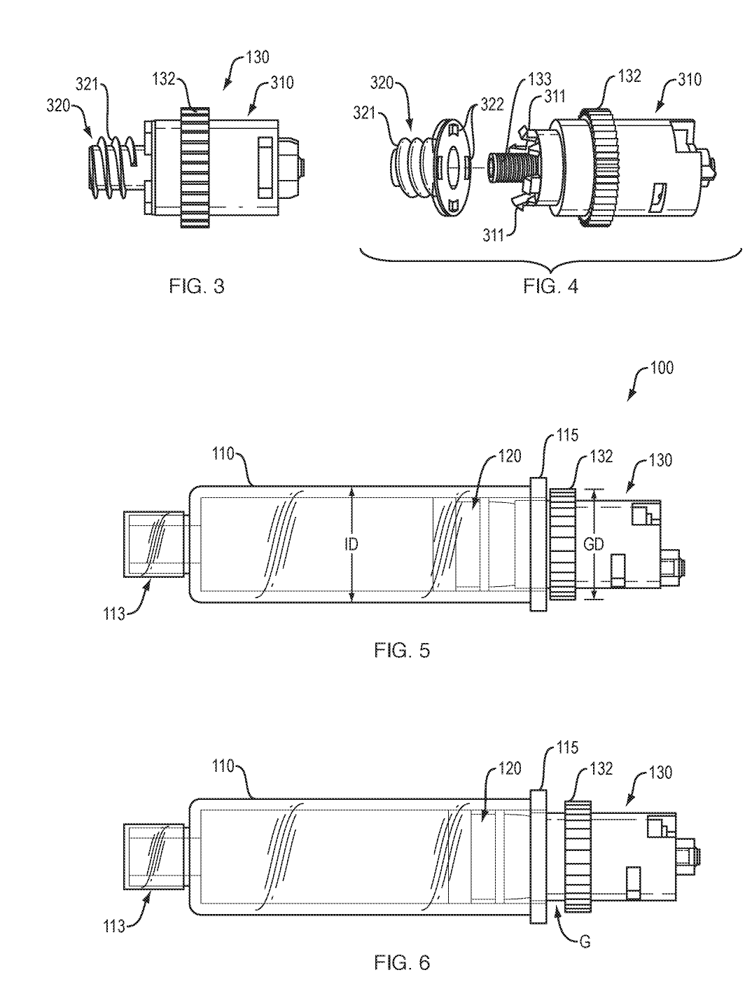

Referring now to FIGS. 3-4, the driving assembly 130 is shown separately in an assembled form (FIG. 3) and a partially dissembled form (FIG. 4). As can be seen, the driving assembly 130 can have a main portion 310 including the gear ring 132 and housing the extendable portion 133 and a stopper connector 320 connected to the extendable portion 133 and having a threading 321 that threads to the stopper 120 in order to connect the stopper 120 to the extendable portion 133. The stopper connector 320 can have a plurality of tab openings 322 formed therein that accept tabs 311 of the extendable portion 133, securing the stopper connector 320 to the extendable portion 133. As the gear ring 132 rotates, thus rotating the rotatable portion 131, the extendable portion 133 and connected stopper connector 320 linearly displace, linearly displacing the connected stopper 120 as well.

As is known, it is critical that proper doses of medicament are delivered to a patient in order to ensure efficacy. One problem that may occur with the delivery assembly 100, and referring now to FIGS. 5-6, occurs when the cartridge 110 and stopper 120 are being connected to the driving assembly 130. When the user inserts the cartridge 110 prior to use of the device, the exposed portion of the driving assembly 130 is pushed axially towards the septum 113 in order to engage the cartridge 110 in the device. The gear ring 132 has a larger diameter GD than an inside diameter ID of the cartridge 110, so if the driving assembly 130 is pushed axially with the ring gear 132 in contact with the cartridge 110, the force is transmitted directly to the cartridge 110. However, the positioning of the stopper 120 after filling of the cartridge 110 is subject to an axial tolerance of, typically, ±1 mm Therefore, the position of the gear ring 132 relative to the cartridge 110 has to take account of the variability of the stopper position and, with the ±1 mm tolerance, this would have to allow for a gap G of up to 2 mm, as shown in FIG. 6. In other words, with the stopper 120 at its deepest position (shown in FIG. 5) the gear ring 132 would be just in contact with the rim 115 of the cartridge 110, and with the stopper 120 at its shallowest position (shown in FIG. 6) there would be a gap G of, in some embodiments, 2 mm between the gear ring 132 and the rim 115 of the cartridge 110.

If there is a gap between the gear ring 132 and the cartridge 110, the axial load on the driving assembly 130 must be transmitted to the cartridge 110 through the interface of the stopper 120 and the cartridge 110, which is resisted only by the friction between these two parts. It is highly likely that the applied force exceeds the frictional force and so the stopper 120 moves towards the septum 113 until the gear ring 132 contacts the cartridge 110, which, when the medicament in the cartridge 110 is a fluid, pressurizes the fluid medicament of the cartridge 110. As the septum 113 is pierced and connected to a fluid delivery path, such as a fluid delivery line connected to a delivery needle, in the same action, the higher pressure inside the cartridge 110 causes the fluid medicament to flow into the fluid path and out of the delivery needle before the needle has penetrated the skin of the patient and is therefore wasted. Further, a user may believe that there is a problem with the device upon noticing the wasted fluid delivered through the fluid path. For a 2 mm movement of the stopper 120, and with a cartridge inside diameter ID of 12.27 mm, a volume of up to 0.236 mL can be lost, which can represent roughly 10% of the medicament volume. This, in turn, means that the cartridge 110 has to be over-filled by a similar volume to compensate for the potential loss, and also that the accuracy of the delivered dose is compromised by the volume of the over-fill because it may or may not be delivered, depending on the size of the gap G between the gear ring 132 and the cartridge 110 before use, and whether or not the stopper friction is overcome during loading of the cartridge 110.

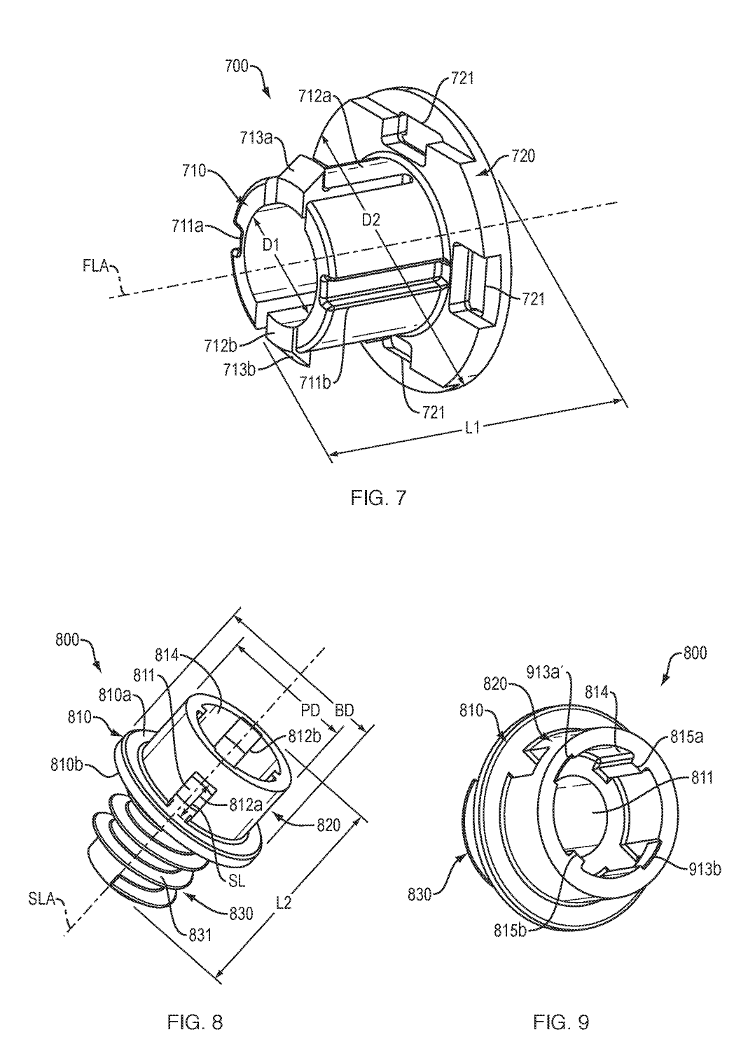

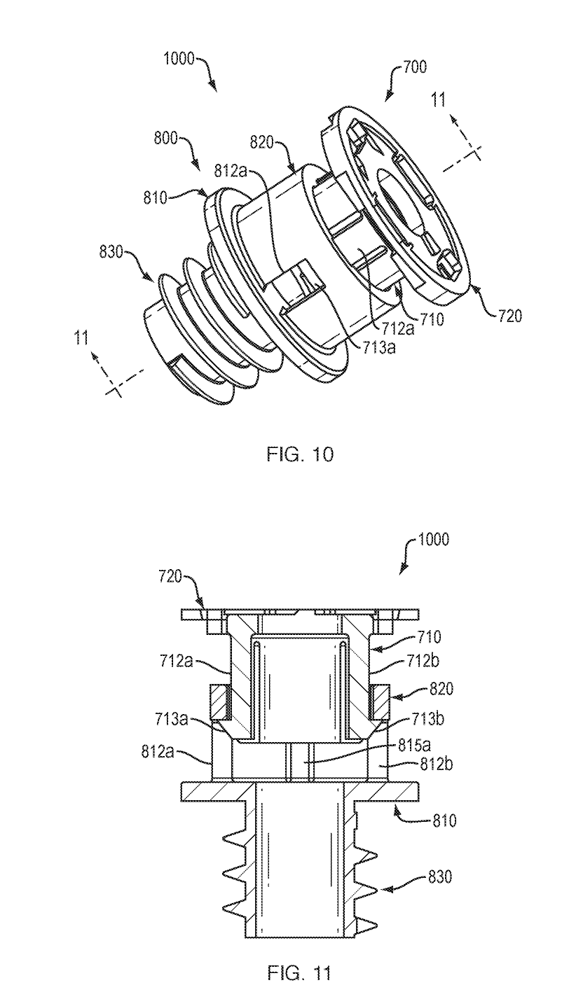

Having identified the previously described issue with the delivery assembly 100 shown in FIGS. 1-6, and referring now to FIGS. 7-13, exemplary embodiments formed in accordance with the present invention provide a connector assembly 1000 (shown assembled in FIGS. 10-13) including a first connector portion 700 (shown individually in FIG. 7) for connecting to the expandable portion 133 and a second connector portion 800 (shown individually in FIGS. 8-9) for connecting to the stopper 120, i.e., the connector assembly 1000 replaces the stopper connector 320. The first connector portion 700 and second connector portion 800 are rotationally locked and axially displaceable relative to one another, the significance of which will be described further herein.

Referring specifically now to FIG. 7, the first connector portion 700 is shown individually and can include a main cylindrical portion 710 connected to an interfacing portion 720. As can be seen, the first connector portion 700 can define a first longitudinal axis FLA and have multiple diameters defined about the first longitudinal axis FLA. The main cylindrical portion 710 may, in some exemplary embodiments, have a cylindrical shape with a first diameter D1 and the interfacing portion 720 may have an annular shape with a second diameter D2 that is greater than the first diameter D1. In some exemplary embodiments, the main cylindrical portion 710 can have one or more locking grooves, shown as a pair of locking grooves 711A and 711B, which extend along the first longitudinal axis FLA across an entire length L1 of the main cylindrical portion 710. As shown, the locking grooves 711A, 711B may be spaced apart 180° about the first longitudinal axis FLA, but it should be appreciated that such spacing is optional. The main cylindrical portion 710 may also include one or more sliding protrusions, shown as a pair of sliding tabs 712A, 712B, that can extend along the first longitudinal axis FLA across most of, but not the entire, length L1 of the main cylindrical portion 710. Each sliding protrusion 712A, 712B may be formed, for example, as a leg from the main cylindrical portion 710 and include a respective chamfer 713A, 713B, the significance of which will be described further herein. The sliding protrusions 712A, 712B may also be spaced apart 180° about the first longitudinal axis FLA, but it should be appreciated that such spacing is optional, and placed in between the locking grooves 711A, 711B, as shown. The interfacing portion 720 may include a plurality of tab openings 721 to accept, for example, tabs 311 of the extendable portion 133, similar to the previously described stopper connector 320.

Referring specifically now to FIGS. 8-9, the second connector portion 800 is shown individually and can include a base 810 with a sliding portion 820 and a threaded portion 830 connected thereto, with the sliding portion 820 being on a first side 810A of the base 810 and the threaded portion 830 on a second side 810B of the base 810 opposite the first side 810A. The second connector portion 800 can define a second longitudinal axis SLA and a second length L2, with the base 810 having a generally annular shape defined about the second longitudinal axis SLA with a base diameter BD. In some exemplary embodiments, the base 810 and threaded portion 830 have an opening 811 extending therethrough. The sliding portion 820, as shown, can have a generally cylindrical shape defined about the second longitudinal axis SLA and have a portion diameter PD less than the base diameter BD. As can be seen, the sliding portion 820 can have one or more sliding slots, shown as a pair of sliding slots 812A and 812B, formed therein that are sized to accept the sliding protrusions 712A, 712B of the first connector portion 700, as will be described further herein. The sliding slots 812A, 812B can be formed in the second connector portion 800 to correspond to the locations of the sliding protrusions 712A, 712B when the connector assembly 1000 is assembled, as can be seen in FIG. 10. Each sliding slot 812A, 812B can define a respective slot length SL that determines the distance the sliding protrusions 712A, 712B can slide within the slots 812A, 812B, as will be described further herein. The sliding portion 820 can also include one or more assembly grooves 913A, 913B formed on an inner surface 814 of the sliding portion 820 and aligned with each of the sliding slots 812A, 812B to allow assembly of the connector assembly 1000, as will be described further herein. In some exemplary embodiments, one or more locking protrusions, shown as a pair of locking splines 815A, 815B, are formed on the inner surface 814 of the sliding portion 820 that fit within the locking grooves 711A, 711B to rotationally lock the first connector portion 700 and second connector portion 800. The locking protrusions 815A, 815B can be spaced apart to align with the locking grooves 711A, 711B during assembly and have a respective protrusion length matching the length L1 of the locking grooves 711A, 711B. The threaded portion 830 can have threading 831 that is similar to the previously described stopper connector 320, which can be adjusted as desired. While the second connector portion 800 is shown as being a separate piece from the stopper 120, it should be appreciated that some or all of the second connector portion 800, such as the base 810 and sliding portion 820, can be integrally formed with the stopper 120, if desired.

Referring specifically now to FIGS. 10-13, the connector assembly 1000 is shown in an assembled state with the first connector portion 700 and the second connector portion 800 telescopically connected to one another, i.e., the first connector portion 700 and the second connector portion 800 are both rotationally locked and axially slideable relative to one another. In some exemplary embodiments, the connector portions 700, 800 can be formed of a plastic material, such as acetal, by molding, allowing for economical manufacturing of the connector portions 700, 800.

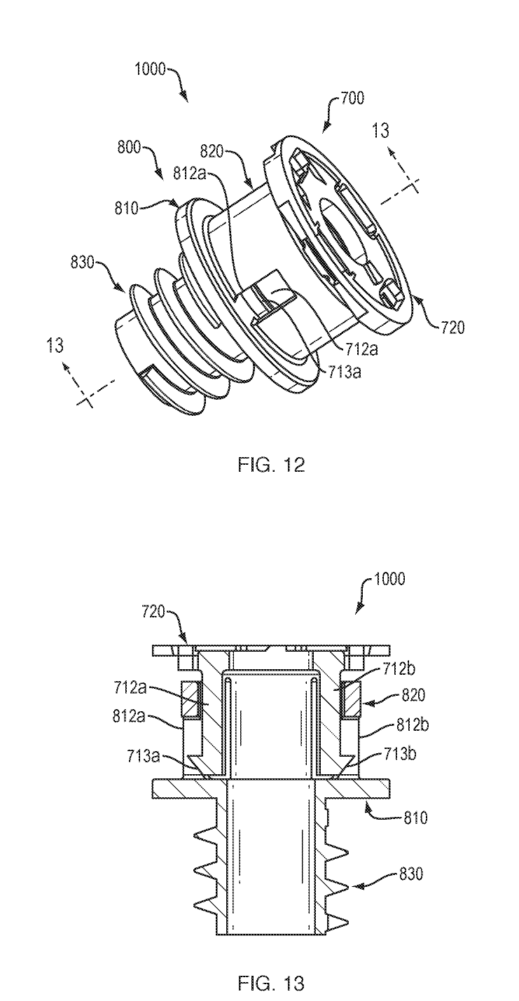

The two connector portions 700, 800 can be coupled by inserting the main cylindrical portion 710 of the connector 700 into the sliding portion 820 of the second connector portion 800 so that the locking splines 815A, 815B of the second connector portion 800 engage with the locking grooves 711A, 711B of the first connector portion 700. The sliding tabs 712A, 712B of the first connector portion 700 can deflect radially inwards elastically during insertion, driven by the interaction of the leading chamfers 713A, 713B acting on the inner surface 814 of the sliding portion 820 of the second connector portion 800, and then snap back out into their original position when they align with the sliding slots 812A, 812B in the sliding portion 820 of the second connector portion 800. At this point, surfaces of the sliding tabs 712A, 712B are presented to the inside surface of the slots 812A, 812B, distal to the threading 831 in such a manner that axial separation of the two connector portions 700, 800 is prevented by the engagement of these surfaces, as illustrated in FIGS. 10-11. When the connector portions 700, 800 are in the relative positions shown in FIGS. 10-11, the connector assembly 1000 can be referred to as being in an “extended position.” The limit of relative axial motion of the two connector portions 700, 800 in the opposite direction is determined by contact of the end surface of the main cylindrical portion 710 of the first connector portion 700 with the surface of the base 810 of the second connector portion 800, as illustrated in FIGS. 12-13. When the connector portions 700, 800 are in the relative positions shown in FIGS. 12-13, the connector assembly 1000 can be referred to as being in a “compressed position.”

The distance by which the two connector portions 700, 800 can move axially relative to each other, i.e., the slot length SL, is arranged to be equal to the total tolerance of the stopper positioning. Thus, where the stopper positioning tolerance is ±1 mm, the relative axial movement of the two connector portions 700, 800 is 2 mm Furthermore, the respective lengths L1, L2 of the two connector portions 700, 800 are arranged such that when the stopper 120 is at the limit of its position that is furthest from the second open end 114 of the cartridge 110, the connector assembly 1000 is in the extended position and the gear ring 132 is in contact with the cartridge 110. Thus also, when the stopper 120 is at the limit of its position that is nearest to the second open end 114 of the cartridge 110, the connector assembly 1000 is free to move from the extended position to the compressed position without transmission of any load to the stopper 120 because the gear ring 132 contacts the cartridge 110 at the same time that the compressed position of the connector assembly 1000 is achieved. This means that no compression of the fluid in the cartridge volume 111 occurs, therefore no loss of fluid when the cartridge 110 is connected to a fluid delivery path, and hence no requirement to over-fill the cartridge 100 as previously described. In this sense, the previously described connector assembly 1000 can counteract the effects of differing positions of the stopper 120 within the cartridge 110. Preventing the fluid loss due to pressurization not only prevents expensive medicaments, such as biologic medicaments, from being wasted, but can also increase the accuracy of the administered dose and prevent false-positive malfunction reports that occur when the user notices the medicament being wasted and believes there is a malfunction with the device.

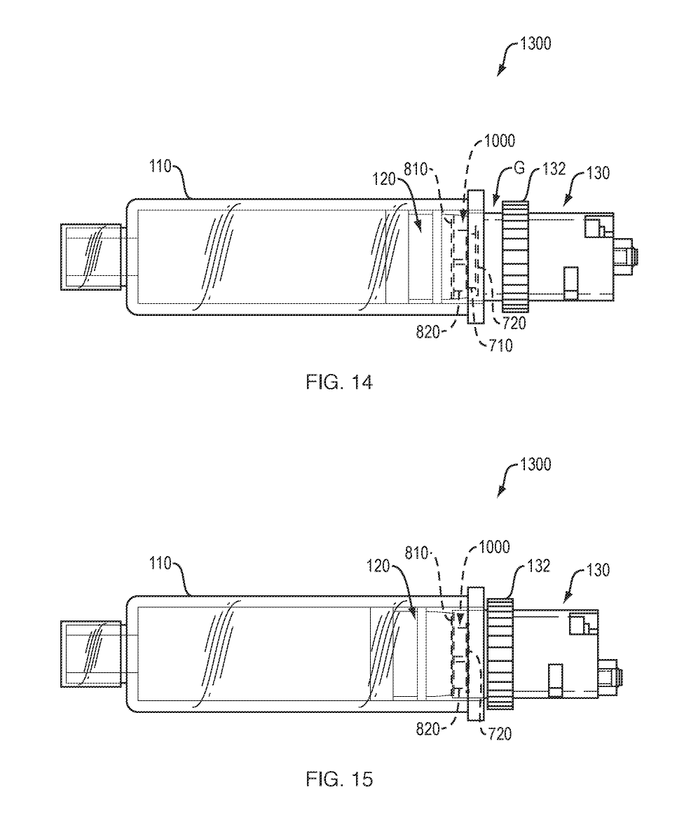

Referring now to FIGS. 14-15, a delivery assembly 1300 is shown that is similar to the previously described delivery assembly 100 but includes the connector assembly 1000 used in place of the stopper connector 320 to connect the stopper 120 to the driving assembly 130. As can be seen in FIG. 14, when the stopper 120 is in its shallowest position within the cartridge 110, a gap G is formed between the gear ring 132 and the cartridge 110, similarly to what is shown in FIG. 6. Since the connector assembly 1000 has connector portions 700, 800 that can axially displace relative to one another, the connector assembly 1000 is initially in the extended position when the stopper 120 is in its shallowest position and the gap G is formed. When axial load is applied to the driving assembly 130 and the gap G closes when the gear ring 132 contact the cartridge 110, as shown in FIG. 15, the first connector portion 700 axially slides within the second connector portion 800 so the connector assembly 1000 assumes the compressed position, allowing for closure of the gap G without displacing the stopper 120 within the cartridge 110, i.e., the stopper 120 maintains its original position when the gap G closes. When the gap G closes, axial displacement of the first connector portion 700 relative to the second connector portion 800 takes place instead of axial displacement of the stopper 120 so the medicament in the cartridge 110 is not pressurized and will not be wasted when the septum 113 is pierced to connect the cartridge 110 to a fluid delivery path.

While the previously described connector assembly 1000 has been particularly described and illustrated for use in delivery devices such as drug pumps, exemplary embodiments of connector assemblies formed in accordance with the present invention can be used in automatic injector devices. In some exemplary embodiments, the connector assembly can be used in addition to, or in place of, the actuation member or plunger of the automatic injector. When used in automatic injector devices, the connector assembly can also counteract the effects of tolerance stack up affecting the initial position of the plunger in order to reduce the need for over-filling, increase the accuracy of delivered doses, and prevent medicament being wasted prior to injection.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step to may be replaced with a plurality of elements or steps that serve the same purpose. Further, where parameters for various properties are specified herein for exemplary embodiments, those parameters may be adjusted up or down by 1/20th, 1/10^{th}, ⅕th, ⅓rd, ½nd, and the like, or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

1. A delivery device for delivering a medicament to a patient, comprising:
a cartridge disposable within a housing having a first end sealed by a septum and a second open end longitudinally opposed to the first end and defining a cartridge volume for holding the medicament;
a stopper at least partially disposed within the cartridge volume and displaceable within the cartridge volume to eject the medicament from the cartridge through the septum;
a driving assembly connected to the stopper having a rotatable portion cooperatively engaged to an extendable portion such that rotation of the rotatable portion causes axial translation of the extendable portion; and
a connector assembly having a first connector portion connected to the extendable portion and a second connector portion connected to the stopper, the first connector portion and second connector portion being rotationally locked and axially displaceable relative to one another.

2. The delivery device of claim 1, wherein the first connector portion and second connector portion are axially slideable relative to one another.

3. The delivery device of claim 2, wherein one of the first connector portion and second connector portion has at least one locking groove formed therein and the other one of the first connector portion and second connector portion has at least one locking protrusion disposed in the at least one locking groove to rotationally lock the first connector portion and second connector portion.

4. The delivery device of claim 2, wherein one of the first connector portion and second connector portion has at least one sliding slot formed therein and the other one of the first connector portion and second connector portion has at least one sliding protrusion slideably held within the at least one sliding slot.

5. The delivery device of claim 4, wherein the at least one sliding protrusion includes a chamfer.

6. The delivery device of claim 1, wherein the first connector portion and second connector portion are telescopically connected to one another.

7. The delivery device of claim 1, wherein the first connector portion and second connector portion axially displace relative to one another when an axial load is applied to at least one of the driving assembly and the cartridge.

8. The delivery device of claim 1, wherein the connector assembly prevents pressurization of the medicament when a gap between the driving assembly and the cartridge closes.

9. The delivery device of claim 1, further comprising a housing that houses the cartridge and driving assembly.

10. A connector assembly for connecting a stopper to a driving assembly in a delivery device, comprising:
a first connector portion for connecting to the driving assembly; and
a second connector portion for connecting to the stopper rotationally locked to the first connector portion and axially displaceable relative to the first connector.

11. The connector assembly of claim 10, wherein the first connector portion and second connector portion are axially slideable relative to one another.

12. The connector assembly of claim 11, wherein one of the first connector portion and second connector portion has at least one locking groove formed therein and the other one of the first connector portion and second connector portion has at least one locking protrusion disposed in the at least one locking groove to rotationally lock the first connector portion and second connector.

13. The connector assembly of claim 11, wherein one of the first connector portion and second connector portion has at least one sliding slot formed therein and the other one of the first connector portion and second connector portion has at least one sliding protrusion slideably held within the at least one sliding slot.

14. The connector assembly of claim 13, wherein the at least one sliding protrusion includes a chamfer

15. The connector assembly of claim 10, wherein the first connector portion and second connector portion are telescopically connected to one another.
